(19) United States
(12) Patent Application Publication
(10) Pub. No.: US 2025/0069295 A1
(43) Pub. Date: Feb. 27, 2025

(54) PROGRESS DIAGRAM GENERATION APPARATUS, PROGRESS DIAGRAM GENERATING METHOD, PROGRAM, AND RECORDING MEDIUM

(71) Applicants: NEC Solution Innovators, Ltd., Tokyo (JP); NATIONAL UNIVERSITY CORPORATION OKAYAMA UNIVERSITY, Okayama-shi, Okayama (JP)

(72) Inventors: Seiko OANA, Tokyo (JP); Yasushi TAKAHASHI, Tokyo (JP); Tsuyoshi MATANO, Tokyo (JP); Ryuichi NAKAHARA, Okayama-shi, Okayama (JP); Keiichiro NISHIDA, Okayama-shi, Okayama (JP); Toshifumi OZAKI, Okayama-shi, Okayama (JP); Yoshihisa NASU, Okayama-shi, Okayama (JP)

(73) Assignees: NEC Solution Innovators, Ltd., Tokyo (JP); NATIONAL UNIVERSITY CORPORATION OKAYAMA UNIVERSITY, Okayama-shi, Okayama (JP)

(21) Appl. No.: 18/724,476

(22) PCT Filed: Oct. 27, 2022
(86) PCT No.: PCT/JP2022/040238

§ 371 Date: Jun. 26, 2024

(30) Foreign Application Priority Data
Dec. 28, 2021 (JP) 2021-214645

(51) Int. Cl. G06T 11/20; G06F 40/242; G06V 30/19

(52) U.S. Cl. CPC G06T 11/206; G06F 40/242; G06V 30/19

(57) ABSTRACT

A progress diagram generation apparatus that allows time series to be easily grasped and input contents to be easily checked is provided. In a progress diagram generation apparatus (100) according to an example aspect of the invention, an observation information acquisition unit (101) is configured to acquire observation information related to an observation target, a time series data generation unit (102) is configured to extract date and time information indicating at least one of a date or a time and event information associated with the date and time information from the observation information and structure them to generate time series data, and a progress diagram generation unit (103) is configured to generate a progress diagram including a first axis indicating a time elapsed and a second axis indicating the event information.

### TECHNICAL FIELD

The present invention relates to a progress diagram generation apparatus, a progress diagram generating method, a program, and a recording medium.

### BACKGROUND ART

Patent Literature 1 discloses an electronic health record system including: a first storage configured to store medical record data for each patient; and a second storage configured to store specific data related to the patient, which is different from the medical record data; a search unit configured to search, in response to an instruction for displaying the medical record data, the second storage for specific data related to the same patient as the medical record data designated by the display instruction; and a display configured to, when the search unit detects corresponding specific data, display the medical record data designated by the display instruction and display the detected specific data or an image indicating that the specific data is present.

### CITATION LIST

### Patent Literature

Patent Literature 1: JP 2005-251082 A

### SUMMARY

### Technical Problem

However, since information to be input is increased by enabling information sharing as in the electronic health record system, the amount of time required for grasping a time series and the amount of work to check for errors in input contents are increased. Therefore, there is a need for a technique to facilitate collection of time series data and checking of input contents. This problem is not only a problem in a medical field, but also a general problem in the field of observing a specific observation object.

With the foregoing in mind, an example object of the invention is to provide a progress diagram generation apparatus that facilitate the works of grasping time series and checking input contents, a progress diagram generating method using the progress diagram generation apparatus, a program for the progress diagram generation apparatus, and a recording medium recorded with the program.

### Solution to Problem

In order to achieve the above object, a progress diagram generation apparatus according to an example aspect of the invention includes:
an observation information acquisition unit;
a time series data generation unit; and
a progress diagram generation unit, wherein
the observation information acquisition unit is configured to acquire observation information related to an observation target,
the time series data generation unit is configured to extract date and time information indicating at least one of a date or a time and event information associated with the date and time information from the observation information and structure them to generate time series data, and
the progress diagram generation unit is configured to generate a progress diagram showing the event information along a time elapsed based on the time series data.

A progress diagram generating method according to an example aspect of the invention includes:
observation information acquiring;
time series data generating; and
progress diagram generating, wherein
the observation information acquiring acquires observation information related to an observation target,
the time series data generating extracts date and time information indicating at least one of a date or a time and event information associated with the date and time information from the observation information and structures them to generate time series data, and
the progress diagram generating generates a progress diagram showing the event information along a time elapsed based on the time series data.

### Advantageous Effects of Invention

An example advantage according to the invention is that time series can be easily grasped and input contents can be easily checked.

### BRIEF DESCRIPTION OF DRAWINGS

FIG. 1 is a block diagram showing an example of a configuration of a progress diagram generation apparatus according to the first example embodiment.

FIG. 2 is a block diagram showing an example of a hardware configuration of a progress diagram generation apparatus according to the first example embodiment.

FIG. 3 is a flowchart showing an example of processing in the progress diagram generating method according to the first example embodiment.

FIG. 4 is a schematic diagram showing an example of the observation information and the progress diagram displayed on a display (user interface) of the progress diagram generation apparatus according to the second example embodiment.

FIG. 5A is a schematic diagram showing an example of the progress diagram in a case where there is an erroneous input in the event information.

FIG. 5B is a schematic diagram showing an example of the progress diagram in a case where there is an erroneous input in the event information.

FIG. 6 is a schematic diagram showing an example of the progress diagram in a case where the date and time is a future date and time.

FIG. 7 is a schematic diagram showing an example of an operation screen (graphical user interface) for implementing each function.

### EXAMPLE EMBODIMENTS

In the progress diagram generation apparatus according to an example aspect of the invention, for example, the progress diagram generation unit may be configured to generate a progress diagram including a first axis indicating a time elapsed and a second axis indicating the event information based on the time series data.

In the progress diagram generation apparatus according to an example aspect of the invention, for example, the observation information acquisition unit may be configured to acquire image information and acquire the observation information from the image information using a character recognition technique.

In the progress diagram generation apparatus according to an example aspect of the invention, for example, the progress diagram generation unit may be configured to generate the progress diagram showing the event information by at least one of a stacked chart, a line chart, or a comment.

In the progress diagram generation apparatus according to an example aspect of the invention, for example, the observation information may include at least one of quantitative information or qualitative information as the event information, and the progress diagram generation unit may be configured to generate the progress diagram showing the quantitative information by at least one of the stacked chart or the line chart and showing the qualitative information by the comment, based on the time series data.

In the progress diagram generation apparatus according to an example aspect of the invention, for example, when the observation information includes date and time information indicating at least one of a date or a time in future later than an execution date and time of processing related to generation of the progress diagram, an object indicating the execution date and time may be drawn in the progress diagram generated by the progress diagram generation unit.

A progress diagram generation apparatus according to an example aspect of the invention may further include, for example, a registration unit, wherein the registration unit may be configured to register a freely selected word to be recognized by the time series data generation unit and classification information in association with each other in dictionary information for recognition, the classification information may be information indicating that the freely selected word is a word classified into any one of the quantitative information to be shown by a stacked chart, the quantitative information to be shown by a line chart, or the qualitative information to be shown by a comment in a progress diagram, and the progress diagram generation unit may be configured to recognize, using the dictionary information, event information having the same word as a word registered in the dictionary information to generate the progress diagram showing the event information.

The progress diagram generation apparatus according to an example aspect of the invention may further include, for example, an output unit, wherein the output unit may be configured to output the progress diagram in an editable format.

In the progress diagram generation apparatus according to an example aspect of the invention, for example, at least one of the observation information acquisition unit, the time series data generation unit, or the progress diagram generation unit may function as an add-in or an application.

In the progress diagram generating method according to an example aspect of the invention, for example, the progress diagram generating may generate a progress diagram including a first axis indicating a time elapsed and a second axis indicating the event information based on the time series data.

In the progress diagram generating method according to an example aspect of the invention, for example, the observation information acquiring may acquire image information and acquire the observation information from the image information using a character recognition technique.

In the progress diagram generating method according to an example aspect of the invention, for example, the progress diagram generating may generate the progress diagram showing the event information by at least one of a stacked chart, a line chart, or a comment.

In the progress diagram generating method according to an example aspect of the invention, for example, the observation information may include at least one of quantitative information or qualitative information as the event information, and the progress diagram generating may generate the progress diagram showing the quantitative information by at least one of the stacked chart or the line chart and showing the qualitative information by the comment, based on the time series data.

In the progress diagram generating method according to an example aspect of the invention, for example, when the observation information includes date and time information indicating at least one of a date or a time in future later than an execution date and time of processing related to generation of the progress diagram, an object indicating the execution date and time may be drawn in the progress diagram generated by the progress diagram generating.

The progress diagram generating method according to an example aspect of the invention may further include, for example, registering, wherein the registering may register a freely selected word to be recognized by the time series data generating and classification information in association with each other in dictionary information for recognition, the classification information may be information indicating that the freely selected word is a word classified into any one of the quantitative information to be shown by a stacked chart, the quantitative information to be shown by a line chart, or the qualitative information to be shown by a comment in a progress diagram, and the progress diagram generating may recognize, using the dictionary information, event information having the same word as a word registered in the dictionary information to generate the progress diagram showing the event information.

The progress diagram generating method according to an example aspect of the invention may further include, for example, outputting, wherein the outputting may output the progress diagram in an editable format.

In the progress diagram generating method according to an example aspect of the invention, for example, at least one of the observation information acquiring, the time series data generating, or the progress diagram generating may function as an add-in or an application.

The program according to an example aspect of the invention is a program for causing a computer to execute processes of the method according to an example aspect of the invention as a procedure.

The recording medium according to an example aspect of the invention is a non-transitory computer readable recording medium recorded with the program according to an example aspect of the invention.

As used herein, the term “observation target” is not particularly limited, and examples thereof include patients, care receivers, toddlers, children, pupils, students, and pets.

An example aspect of the invention is applicable, for example, to fields of medical care, nursing, caring, pet hotels, and educational facilities (such as nursery schools) in which an observation target is present.

Example embodiments of the invention will be described with reference to the drawings. Note here that the invention is not limited to the following example embodiments. In the drawings, identical parts are indicated with identical reference signs. Regarding the descriptions of the example embodiments, reference can be made to one another and the configurations of the example embodiments can be combined, unless otherwise stated. As used herein, the term “unit” may also include, for example, a combination of hardware resources implemented by a circuit in a broad sense and information processing of software that can be specifically executed by these hardware resources.

### First Example Embodiment

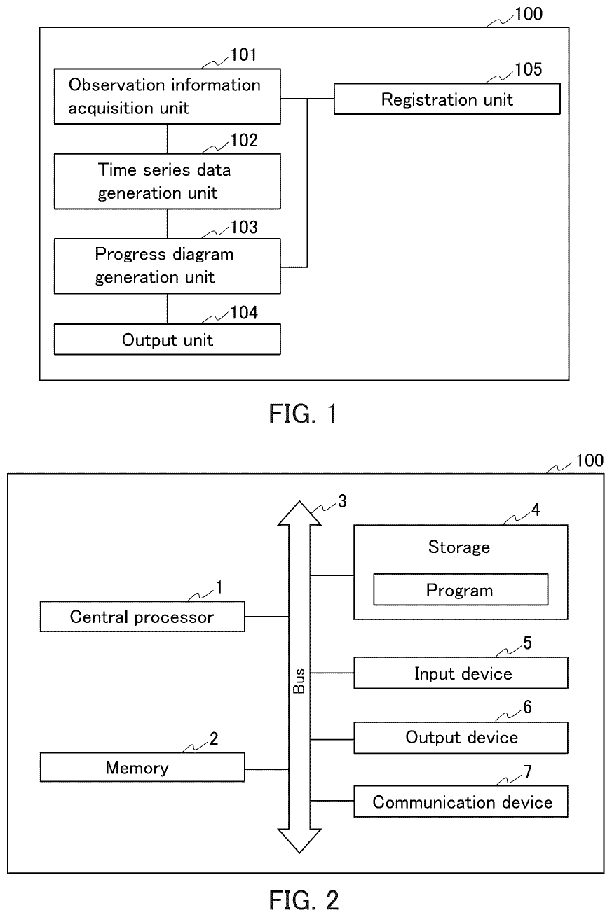

FIG. 1 is a block diagram showing an example of a configuration of a progress diagram generation apparatus 100 according to the present example embodiment. As shown in FIG. 1, the apparatus 100 includes an observation information acquisition unit 101, a time series data generation unit 102, and a progress diagram generation unit 103. The apparatus 100 may further include an output unit 104, a registration unit 105, and the like as optional components.

The apparatus 100 may be, for example, a single apparatus including the respective components, or may be an apparatus to which the respective components are connectable via a communication network. The components may be connected to each other by, for example, an internal bus.

Further, the apparatus 100 can be connected to the external device to be described below via the communication network. The communication network is not particularly limited, and a known network can be used, and may be, for example, a wired network or a wireless network. Examples of the communication network include an internet line, a world wide web (WWW), a telephone line, a local area network (LAN), a storage area network (SAN), a delay tolerant networking (DTN), a low power wide area (LPWA), and a local 5G (L5G). Examples of the wireless communication include WI-FIR, BLUETOOTH®, local 5G, and LPWA. The wireless communication may be direct communication (Ad Hoc communication), infrastructure communication, or indirect communication via an access point.

The apparatus 100 may be incorporated in a server as a system. Further, the apparatus 100 may be, for example, a personal computer (PC, for example, desktop computer, laptop computer), a smartphone, a tablet terminal, a wearable terminal, or the like in which a program according to an example aspect of the invention or an application including a program according to an example aspect of the invention is installed. The apparatus 100 can implement the functions of the respective components by, for example, an application (a program according to an example aspect of the invention or the like) loaded on the main memory. Furthermore, all or some of the components of the apparatus 100 may be implemented on the cloud. Specifically, the apparatus 100 may be in the form of cloud computing, edge computing, or the like, for example, in which at least one of the components is located on a server (cloud) and the other components are located on a terminal.

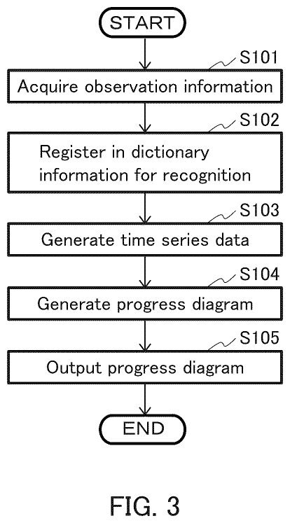

FIG. 2 is a block diagram showing the hardware configuration of the apparatus 100. The apparatus 100 may include, for example, a central processing unit (such as a CPU, GPU) 1, a memory 2, a bus 3, a storage 4, an input device 5, an output device 6, a communication device 7, and the like. Note that these are examples, and the hardware configuration of the apparatus 100 is not limited to this as long as the processing of each component can be executed. In addition, the number of the central processing units 1 and the like included in the apparatus 100 is not limited to the example shown in FIG. 2, and for example, a plurality of central processing units 1 may be included in the apparatus 100. The components of the hardware configuration of the apparatus 100 are connected to each other via the bus 3 by respective interfaces (I/F).

The central processing unit 1 is responsible for overall control of the apparatus 100. In the apparatus 100, the central processing unit 1 executes, for example, a program according to an example aspect of the invention and other programs, and reads and writes various kinds of information. The central processing unit 1 functions as, for example, an observation information acquisition unit 101, a time series data generation unit 102, a progress diagram generation unit 103, an output unit 104, and a registration unit 105.

The bus 3 can also be connected to an external device, for example. Examples of the external device include an external storage (such as an external database), an external input device, and an external output device. The apparatus 100 can be connected to an external network (the communication network) by, for example, the communication device 7 connected to the bus 3, and can also be connected to other devices via the external network.

The memory 2 is, for example, a main memory (main storage). When the central processing unit 1 performs processing, for example, the memory 2 reads various operation programs such as a program according to an example aspect of the invention stored in the storage 4 to be described below, and the central processing unit 1 receives data from the memory 2 and executes the program. The main memory is, for example, a random access memory (RAM). The memory 2 may be, for example, a read-only memory (ROM).

The storage 4 is also referred to as, for example, a so-called auxiliary storage with respect to the main memory (main storage). As described above, the storage 4 stores operation programs including the program of an example aspect the invention. The storage 4 may be, for example, a combination of a recording medium and a drive that reads from and writes to the recording medium. The recording medium is not particularly limited, and may be, for example, a built-in type or an external type, and examples thereof include hard disks (HDs), CD-ROMs, CD-R's, CD-RWs, MOs, DVDs, flash memories, and memory cards. The storage 4 may be, for example, a hard disk drive (HDD) in which a recording medium and a drive are integrated, or a solid state drive (SSD).

In the apparatus 100, the memory 2 and the storage 4 can also store various kinds of information such as log information, information acquired from an external database (not shown) or an external device, information generated by each processing of the apparatus 100, and information used when the apparatus 100 executes each processing. Note that at least some pieces of information may be stored in an external server other than the memory 2 or the storage 4, or may be distributed and stored in a plurality of terminals using a blockchain technology or the like.

The apparatus 100 may further include a user interface, such as an input device 5 and an output device 6. The input device 5 is, for example, a device that inputs characters, numbers, positions of objects displayed on the screen, images, sounds, and the like, and specific examples thereof include a digitizer (a touch panel or the like), a keyboard, a mouse, a scanner, an imaging device, a microphone, and a sensor. Examples of the output device 6 include a display (LED display, liquid crystal display), a printer, and a speaker. The input device 5 and the output device 6 may be, for example, a user interface such as a touch panel in which they are combined.

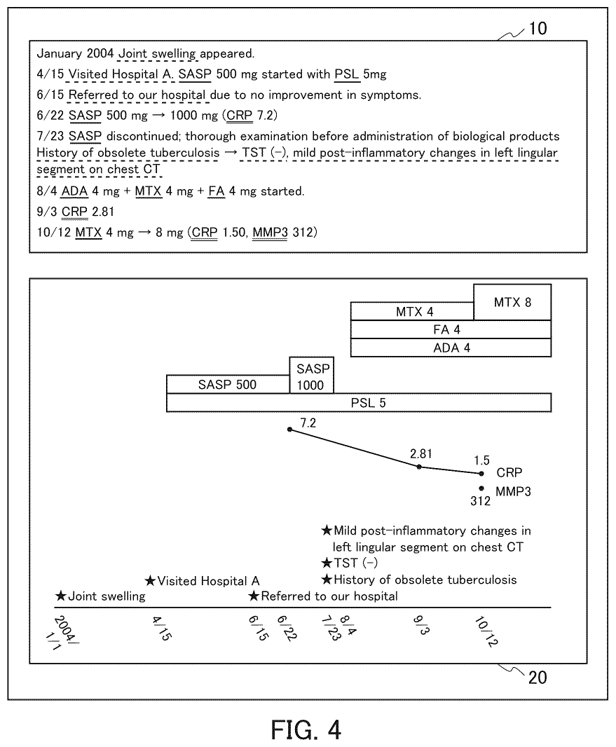

Next, an example of the progress diagram generating method of the present example embodiment will be described based on the flowchart of FIG. 3. The progress diagram generating method of the present example embodiment is performed as follows, for example, using the progress diagram generation apparatus 100 of FIG. 1. Note that the progress diagram generating method of the present example embodiment is not limited to the use of the progress diagram generation apparatus 100 of FIG. 1.

In the progress diagram generating method of the present example embodiment, the observation information acquiring may be performed by, for example, the observation information acquisition unit 101, the time series data generating may be performed by, for example, the time series data generation unit 102, the progress diagram generating may be performed by, for example, the progress diagram generation unit 103, the outputting may be performed by, for example, the output unit 104, and the registering may be performed by, for example, the registration unit 105.

First, the observation information acquisition unit 101 acquires observation information related to an observation target (S101). The observation information is, for example, text data including date and time information indicating at least one of a date and a time (hereinafter, also referred to as a date and time) and event information associated with the date and time information. The date and time may be at least one of a date and a time in future later than an execution date and time of a series of processing related to generation of a progress diagram by the apparatus 100. Hereinafter, at least one of the date and the time in future is also referred to as a future date. In addition to the date and time information and the event information, the observation information may include, for example, attribute information (e.g., identification information, name, gender, belonging organization, age, etc.) of the observation target, information (e.g., identification information, name, belonging organization, etc.) capable of specifying an observer who observes the observation target, information (e.g., identification information, name, belonging organization, etc.) capable of specifying an input person (e.g., the observer, etc.) who has input the event information, and the like. The date may be in any unit of year, month, and day, or may be a combination thereof.

The observation information includes, for example, at least one of quantitative information and qualitative information as the event information. The quantitative information is information that can be expressed numerically, and for example, information accompanied by numeric values such as results of various tests and a dose (prescription amount) of a drug in the medical field. Here, the numerical value may include, for example, not only an actual numerical value but also a word indicating a specific numerical value such as “0”. Examples of such a word include “stop”, “end”, and “half”. The qualitative information is information that cannot be expressed numerically, and for example, information not accompanied by numeric values such as symptoms, medical histories, and behavior histories in the medical field. Whether the word in the event information is the quantitative information or the qualitative information may be specified by, for example, the natural language analysis by the time series data generation unit 102 to be described below, or may be specified by the progress diagram generation unit 103 using dictionary information for recognition to be described below. The apparatus 100 may display the observation information on a display (output device 6) by the output unit 104 to be described below, for example.

The method for acquiring the observation information by the observation information acquisition unit 101 is not particularly limited, and for example, the observation information input by an input device (the input device 5 or an external input device) may be acquired, or the observation information may be acquired from an external device (a server or the like) via the communication network. Further, for example, the observation information acquisition unit 101 may acquire image information and acquire the observation information (text data) from the image information using a character recognition technique. The character recognition technique is not particularly limited, and known techniques such as optical character recognition and the like can be used. For example, the observation information acquisition unit 101 may acquire the image information by the input device 5 or acquire the image information by the communication network in the same manner as the observation information. As described above, by using the character recognition technique, for example, it becomes easy for the user to cause the apparatus 100 to acquire the observation information from another terminal (PC, smartphone, tablet terminal, or wearable terminal).

Next, the registration unit 105 registers a freely selected word to be recognized by the time series data generation unit 102 and the classification information in association with each other in dictionary information for recognition (S102). The classification information is information indicating that the freely selected word is a word classified into any one of the quantitative information to be shown by a stacked chart, the quantitative information to be shown by a line chart, and the qualitative information to be shown by a comment in the progress diagram. For example, the registration unit 105 associates the freely selected word with the classification information by input by the user. The freely selected word registered in the dictionary information for recognition is, for example, a freely selected word that can be shown in a progress diagram, and examples thereof include names such as a test name, a drug name, and the like; and abbreviation and dialects thereof. The registration unit 105 may receive an input of the freely selected word via an input device (the input device 5 or an external input device), or may acquire the freely selected word from an external device, for example. In the former case, the registration unit 105 may register a freely selected word from the event information displayed on the apparatus 100 in association with the classification information in the dictionary information for recognition, for example, by selection by the user via an input device (the input device 5 or an external input device). For example, the registration unit 105 may perform the registration by storing the freely selected word in the memory 2 or the storage 4, or may perform the registration by storing the freely selected word in an external storage. The dictionary information for recognition is information obtained by summarizing the freely selected words registered by the registration unit 105 and the classification information thereof.

While FIG. 3 shows an example in which S102 is executed after S101 and prior to S103 to be described below, an example aspect of the invention is not limited thereto, and S102 can be executed at any time.

For example, the registration unit 105 may register a freely selected word not to be recognized by the time series data generation unit 102, that is, a freely selected word to be not shown by the progress diagram, in the dictionary information for non-recognition. The dictionary information for non-recognition is information obtained by summarizing the freely selected words registered by the registration unit 105. The registration of the dictionary information for non-recognition can be executed, for example, in the same manner as the processing related to the registration of the dictionary information for recognition described above by replacing the “dictionary information for recognition” with “dictionary information for non-recognition”.

Next, the time series data generation unit 102 extracts date and time information indicating at least one of a date and a time and event information associated with the date and time information from the observation information and structures them to generate time series data (S103). That is, it can be said that the time series data generation unit 12 extracts the event information using the date information as a key. The time series data generation unit 12 may perform the extraction by, for example, natural language processing. The structuring is, for example, that the event information classified into the same classification is set as a group of information, and a hierarchical relationship (time series relationship) of the group of information is specified based on the date and time information. The time series data is, for example, data indicating a hierarchical relationship for each group of information. The apparatus 100 may display the time series data on a display (output device 6) by the output unit 104 to be described below, for example.

The structuring by the time series data generation unit 102 will be described in more detail. The time series data generation unit 102 may, for example, classify the event information into the quantitative information or the qualitative information, and the quantitative information and the qualitative information each may be set as the group of information. The event information may be classified by, for example, natural language processing or using the dictionary information for recognition. In addition, the time series data generation unit 102 may subdivide the pieces of quantitative information and set the pieces of quantitative information having the same keyword as the group of information, for example. In the medical field, the keywords are, for example, information (information that is not a numerical value) associated with the numerical value in the quantitative information, specifically, names such as a test name and a drug name. Thereafter, the time series data generation unit 102 specifies a time series relationship of pieces of information of the group of information, for example, based on the date and time information. For example, the time series data generation unit 102 may generate the time series data by estimating that the state of the numerical value indicated by the quantitative information continues as long as there is no change in the numerical value indicated by the quantitative information.

Next, the progress diagram generation unit 103 generates a progress diagram showing the event information along a time elapsed based on the time series data (S104). When S105 to be described below is not executed, the progress diagram generating method according to the present example embodiment may be ended after S104 is executed. For example, the progress diagram generation unit 103 may generate, as the progress diagram, a progress diagram including a first axis indicating a time lapsed and a second axis indicating the event information based on the time series data (it can also be said that the first axis and the second axis are shown in the progress diagram). Further, for example, the progress diagram generation unit 103 may generate the progress diagram showing the event information by at least one of a stacked chart, a line chart, and a comment according to the classification of the event information (it can also be said that each event information is shown in the progress diagram in each format). The classification of the event information is classification based on the above described structuring. That is, the progress diagram is a diagram that shows the event information for each group of information along a time series. Specifically, for example, the progress diagram generation unit 103 generates the progress diagram showing the quantitative information by at least one of the stacked chart and the line chart and showing the qualitative information by the comment, based on the time series data. The apparatus 100 may be configured to display the progress diagram on a display (output device 6) by the output unit 104 to be described below, for example.

When the observation information includes date and time information indicating at least one of a date and a time in future later than an execution date and time of processing related to generation of the progress diagram, an object indicating the execution date and time may be drawn in the progress diagram generated by the progress diagram generation unit 103. Specific examples of the progress diagram in a case where the future date is included will be described below.

The progress diagram generation unit 103 may, for example, recognize, using the dictionary information for recognition, event information having the same word as a word registered in the dictionary information for recognition to generate the progress diagram showing the event information. The event information that does not include a word registered in the dictionary information for recognition may not be shown in the progress diagram. Further, the progress diagram generation unit 103 may, for example, recognize, using the dictionary information for non-recognition, event information having the same word as a word registered in the dictionary information for non-recognition from the observation information to generate the progress diagram not showing the event information (it can also be said that the event information is deleted from the progress diagram).

Then, after S103, the output unit 104 outputs the progress diagram in an editable format (S105) and the procedure is ended (END). Note that the step S105 is optional and may not be performed. The output unit 104 may output the progress diagram to the output device 6 or may output the progress diagram to the outside via the communication network, for example. The editable format is, for example, a format that can be edited by a program (application) other than the program (application) according to an example aspect of the invention. Specific examples of such an editable format include an application for text editing and an application for slide editing. In this way, by outputting the progress diagram in an editable format, it is possible to assist the preparation of materials such as materials for research, conference, and the like. This leads to a reduction in work time by speeding up preparation of materials.

When the apparatus 100 includes the registration unit 105, the observation information acquisition unit 101 may assist the input of the observation information by using at least one of the dictionary information for recognition and the dictionary information for non-recognition before the acquisition of the observation information, for example. The assistance is not particularly limited, and may be, for example, proposing a word when the observation information is input (also referred to as predictive text). By the assistance, for example, it is possible to reduce an erroneous input at the time of inputting the observation information, and it is possible to further improve the accuracy of the contents of the observation information.

For example, the registration unit 105 may register the dictionary information for recognition and the dictionary information for non-recognition in a format that can be shared with other devices. Specifically, the sharing can be achieved by registering the information in a device accessible by another device, a storage (which may be the storage 4) managed by a server, a database, and the like. Thus, for example, it is possible to assist the statistics sharing by team medical care and inter-hospital information cooperation.

According to the present example embodiment, by generating the progress diagram based on the time series data, time series can be easily grasped and input contents can be easily checked. If the works of grasping the time series and checking the input contents become easy, for example, a time for the work (including overtime work) is reduced. In addition, by showing the time series data in the progress diagram. visibility is improved, and it becomes easy to grasp the contents of the observation information, that is, it becomes easy to grasp the outcome of the treatment, nursing care, etc, given to the observation target. Further, the progress diagram allows an erroneous input or the like in the observation information to be easily found and accuracy of the input contents to be improved. If the apparatus 100 is an apparatus in which the program according to an example aspect of the invention or an application including the program according to an example aspect of the invention is installed, it can be used in, for example, a multivendor environment.

### Second Example Embodiment

An example in which an example aspect of the invention is applied to an electronic health record of a medical site will be described. Note here that the invention is not limited to this, and as described above, the invention can be applied to fields other than the medical field.

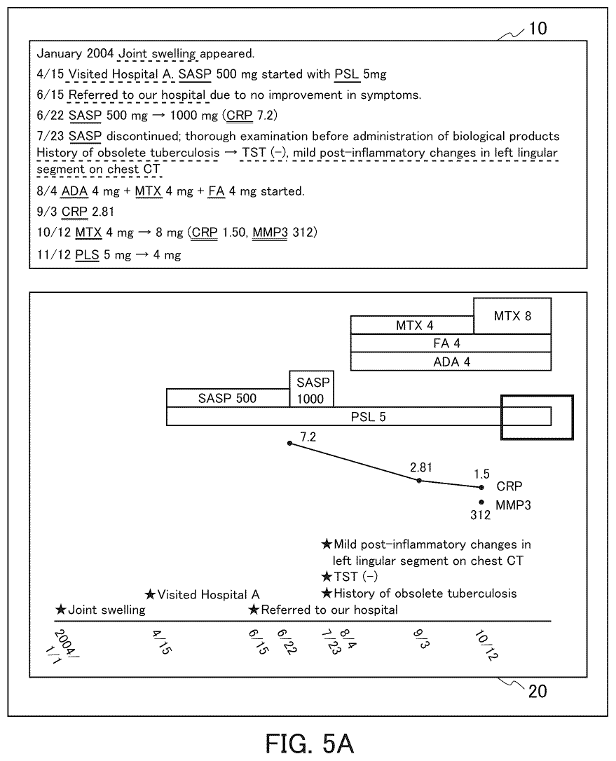

FIG. 4 is a schematic diagram showing an example of the observation information and the progress diagram displayed on a display (user interface) of the apparatus 100. In the present example embodiment, it is assumed that the future date is not included. For example, the observation information acquisition unit 101 acquires the observation information input to an input field 10. The apparatus 100 displays, for example, a progress diagram generated by the progress diagram generation unit 103 in a progress diagram display field 20. In the progress diagram of the present example embodiment, the horizontal axis is the first axis, and the vertical axis is the second axis. As shown in FIG. 4, for example, the apparatus 100 generates (updates) the progress diagram in real time each time text data is input to the input field 10 (that is, each time the observation information is updated).

The input field 10 may be, for example, a field having a fixed height on the display, provided with a scrolling function. The progress diagram display field 20 may be, for example, a field having height variable on the display depending on the size of the progress diagram, not provided with the scrolling function. Note that this is an example, and whether or not the input field 10 and the progress diagram display field 20 are provided with the scrolling function is not limited to the above description.

For example, the progress diagram generation unit 103 may indicate pieces of date information on the first axis in accordance with time intervals. Specifically, as shown in FIG. 4, a gap from 2004 Apr. 15 to 2004 Jun. 15 on the first axis is represented to be wider than a gap from 2004 Jun. 15 to 2004 Jun. 22. As described above, the progress diagram generation unit 103 may set the first axis such that, for example, the longer the time interval is, the wider the gap between corresponding two pieces of date information is, and the shorter the time interval is, the narrower the gap between corresponding two pieces of the date information is. An example of the setting of the first axis by the progress diagram generation unit 103 will be described in more detail. For example, the progress diagram generation unit 103 may define a predetermined length from the start point of the first axis (that is, date and time information indicating the first day in the observation information) as the entire length of the first axis. The length can be set freely according to, for example, the size of the progress diagram display field 20. Then, for example, the progress diagram generation unit 103 may plot date and time information indicating the final day (including the future date) in the observation information at a predetermined point on the first axis, and plot points corresponding to the other pieces of date and time information in accordance with time intervals, between the plot corresponding to the start point and the plot corresponding to the final day. The predetermined point (also referred to as a final day point) is not particularly limited and can be set freely, and is fixed at, for example, 85%, 90%, 95% or the like from the start point toward the end point in the entire length of the first axis. That is, even if the execution date and time fluctuates, the positions of the start point and the final day point on the first axis do not fluctuate, and the positions of the other pieces of date and time information plotted between the start point and the final day point fluctuate in accordance with the execution date and time.

For example, the user may freely select a word among the observation information displayed in the input field 10 using the input device (the input device 5 or the external input device). For example, the registration unit 105 may register the freely selected word in the dictionary information for recognition or the dictionary information for non-recognition in accordance with an operation by the user. Further, the registration unit 105 may, for example, associate the classification information indicating whether the freely selected word is classified into the dictionary information for recognition or the dictionary information for non-recognition in accordance with an operation by the user.

In the present example embodiment, as shown in FIG. 4, it is assumed that, among the observation information input to the input field 10, at least words each indicated with a single underline, a double underline, or a dashed line have been registered in advance as the dictionary information for recognition (hereinafter, the same applies to other drawings unless otherwise specified). Specifically, the registration unit 105 has registered drug names each indicated with a single underline in the dictionary information for recognition as quantitative information shown by a stacked chart, has registered test names each indicated with a double underline in the dictionary information for recognition as quantitative information shown by a line chart, and has registered symptoms and other events each indicated with a dashed line in the dictionary information for recognition as qualitative information shown by a comment.

For example, as shown in FIG. 4, the apparatus 100 may display the observation information by attaching a recognition indicator to a word recognized by the progress diagram generation unit 103. The recognition indicator may be a different indicator for each of the plurality pieces of classification information. While various lines are used as the recognition indicators in FIG. 4, the recognition indicator is not limited thereto, and may be, for example, a marker, graphics, a symbol, or the like.

As shown in FIG. 4, the progress diagram generation unit 103 generates, according to the dictionary information for recognition, a progress diagram showing the dose of the drug (also referred to as the prescription amount) for each drug name by a stacked object (graph), showing the result of the test by a line object (graph) for each test name, and showing each qualitative information by a comment object. As shown in FIG. 4, the progress diagram shows the qualitative information by a comment, the quantitative information by a line chart, and the quantitative information by a stacked chart in this order from a position close to the first axis. That is, the progress diagram includes a display area of quantitative information shown by the stacked chart (drug display area), a display area of quantitative information shown by the line chart (test display area), and a display area of qualitative information shown by a comment (comment display area). The lengths of the display areas in the first axial direction correspond to the length of the first axis. As shown in FIG. 4, the quantitative information shown by the stacked object and the qualitative information shown by the comment object are arranged so as not to overlap with other quantitative information (quantitative information shown by the stacked object) and other qualitative information. On the other hand, the quantitative information shown by the line object may be overlapped with other quantitative information (quantitative information shown by the line object), for example.

For example, as shown in FIG. 4, as to the pieces of quantitative information of the same type, the progress diagram generation unit 103 may show the contents of the pieces of quantitative information of the same type by the stacked chart by using objects each having a different size (length) in the second axial direction in accordance with the difference in the size of the numerical values described subsequent to the dictionary information for recognition. Here, the pieces of quantitative information of the same type means, for example, the pieces of quantitative information in which information other than the numerical value is common (for example, quantitative information including words having the same dictionary information for recognition). That is, for example, if the numerical value of one quantitative information is larger than the numerical value of the other quantitative information, the progress diagram generation unit 103 increases the size of the object corresponding to the one quantitative information to be more than the size of the object corresponding to the other quantitative information. Conversely, for example, if the numerical value of one quantitative information is smaller than the numerical value of the other quantitative information, the progress diagram generation unit 103 reduces the size of the object corresponding to the one quantitative information to be smaller than the size of the object corresponding to the other quantitative information. In the example shown in FIG. 4, since the dose of “SASP” is doubled on 6/22, the size of the object corresponding to “SASP” after 6/22 is increased by a factor of 2 in the second axial direction more than the object corresponding to “SASP” up to 6/22. Note that, while an example in which the size of the object is changed at the same ratio as the difference (ratio) in the magnitude of the numerical value has been described in the present example, the way of the change is not limited thereto. Regarding the numerical values of the pieces of quantitative information and the objects corresponding thereto, it is applicable as long as the magnitude relationship in the numerical values of the pieces of quantitative information corresponds to the magnitude relationship of the objects corresponding thereto.

The progress diagram generation unit 103 may generate the progress diagram, for example, with a position corresponding to the date and time information associated with the quantitative information being set as one end of the stacked object. Then, for example, the progress diagram generation unit 103 may set the other end of the stacked object according to at least one of the following conditions (1) to (3):
(1) When the date and time information associated with the quantitative information is the final day in the observation information, a position having at least a predetermined length from the one end is set as the other end of the stacked object;
(2) When the date and time information associated with the quantitative information is not the final day in the observation information, a position corresponding to the date and time information subsequent to the date and time information associated with the quantitative information is set as the other end of the stacked object; and
(3) When the date and time information associated with the quantitative information is not the final day in the observation information, a position corresponding to the date and time information when the numerical value indicated by the quantitative information fluctuates in accordance with the time series data is set as the other end of the stacked object.

In the condition (1), the predetermined length is not particularly limited, and can be set freely, and is, for example, a length of 3%, 5%, or 7% with respect to the entire length of the first axis. In example of FIG. 4, one end of the object indicating the dose of “MTX” on 10/12, which is the final day of the observation information, is located at a position corresponding to 10/12, and the other end is located at a position having a predetermined length from the one end.

The condition (2) will be described with reference to a case where there is a time series in the order of A, B, and C, there are the pieces of quantitative information shown by the stacked charts in the time series, and C is not the final day of the observation information, as an example. In this example, for example, as to the first object, one end of the object indicating the quantitative information is located at a position corresponding to A and the other end is located at a position corresponding to B. Then, as to the second object, one end of the object indicating the quantitative information is located at a position corresponding to B, and the other end is located at a position corresponding to C. That is, even in the case of the pieces of quantitative information of the same type and when there is no fluctuation in the numerical values indicated by the pieces of quantitative information between A and C, the pieces of quantitative information are shown by individual objects in accordance with the time series.

The condition (3) will be described with reference to FIG. 4 as an example. In FIG. 4, in accordance with the time series of “SASP” which is quantitative information, one end of the first object indicating the dose of “SASP” is located at a position corresponding to 4/15, and the other end is located at a position corresponding to 6/22 on which the dose of “SASP” is changed. Then, one end of the second object indicating the dose of “SASP” is located at a position corresponding to 6/22, and the other end is located at 7/23 on which the administration of “SASP” is stopped. If a numerical value (e.g., “0”) or a word (e.g., “end” or “stop”) indicating “nothing” is associated with the keyword, the position corresponding to the date wish such a numerical value or a word may be set as the other end. On the other hand, in the condition (3), if there is no fluctuation in the numerical value indicated by the quantitative information in the time series data, as shown in FIG. 4, the pieces of quantitative information corresponding to a plurality pieces of date information (the quantitative information shown by the stacked chart) may be represented as one object common to the plurality pieces of date information. That is, the object straddles positions corresponding to the plurality pieces of date information from one end to the other end of the object.

The length of the drug display area in the second axial direction varies depending on, for example, the number of the pieces of quantitative information (also referred to as the number of drugs in the present example embodiment) and the size of the object. For example, the larger the number of the pieces of quantitative information shown by the stacked object, the longer the length of the drug display area in the second axial direction. The apparatus 100 may set a displayable width in the second axial direction for each quantitative information in the drug display area in advance. The displayable width defines a width capable of displaying the quantitative information, in other words, a maximum value of the length of the object in the second axial direction in the pieces of quantitative information of the same type. The width is not particularly limited, and can be set to any width. That is, the progress diagram generation unit 103 may show the quantitative information (specifically, for example, the magnitude relationship of the object) within the range of the displayable width, for example.

The length of the test display area in the second axial direction varies depending on, for example, the number of the pieces of quantitative information shown by the line object (also referred to as the number of test items in the present example embodiment). For example, the larger the number of the pieces of quantitative information, the longer the length of the test display area in the second axial direction. The apparatus 100 may set a displayable width in the second axial direction for each quantitative information in the test display area in advance. The displayable width is the same as described above. That is, the progress diagram generation unit 103 may show the quantitative information, for example, within the range of the displayable width.

The quantitative information shown by the line chart and the qualitative information shown by the comment are shown on positions each corresponding to the date and time information, for example, as shown in FIG. 4. In the case of showing by the comment, for example, as shown in FIG. 4, the comment indicator to be described below may be located on a position corresponding to the date and time information, and the qualitative information may be described subsequent to the comment indicator.

Showing by the comment is describing the qualitative information in the diagram as text data without using a graph or the like. Showing by the comment may be, for example, describing the qualitative information by attaching a comment indicator to the qualitative information. While FIG. 4 shows an example in which a star mark is attached before the qualitative information in the progress diagram, the comment indicator is not limited thereto. In the progress diagram, the qualitative information may be described in one line or in a plurality of lines. Further, as shown in FIG. 4, when a plurality pieces of qualitative information exist at the same date and time in the observation information, the progress diagram generated by the progress diagram generation unit 103 may show the pieces of qualitative information by dividing into a plurality of paragraphs for each piece of qualitative information on a position corresponding to each date and time in the first axis, for example. The length of the comment display area in the second axial direction varies depending on, for example, the number of the pieces of qualitative information.

For example, the numerical value of the quantitative information and information other than the numerical value (word information of the dictionary information for recognition associated with the numerical value) may be described in and around the object of the quantitative information in the progress diagram.

Further, as shown in FIG. 4, when the observation information includes both the date information in the unit including the year (e.g., “January 2004”) and the date information in the unit not including the year (e.g., “4/15”), the time series data generation unit 102 may determine that the year in the date information in the unit not including the year belongs to the year of the date information in the unit including the year described earlier (i.e., “4/15” is “2004 Apr. 15”), for example, according to the description order of the observation information, and may perform the structuring and the like. Also, as to the unit not including the month, for example, the time series data generation unit 102 may determine that the unit belongs to the month of the date information in the unit including the month described earlier in the same manner as the unit of the year described above. Further, for example, as shown in FIG. 4, the time series data generation unit 102 may perform the structuring and the like on the date information (for example, “January 2004”) in the unit not including the date, assuming that the date is the first day of the month (that is, “January 2004” is “2004 Jan. 1”).

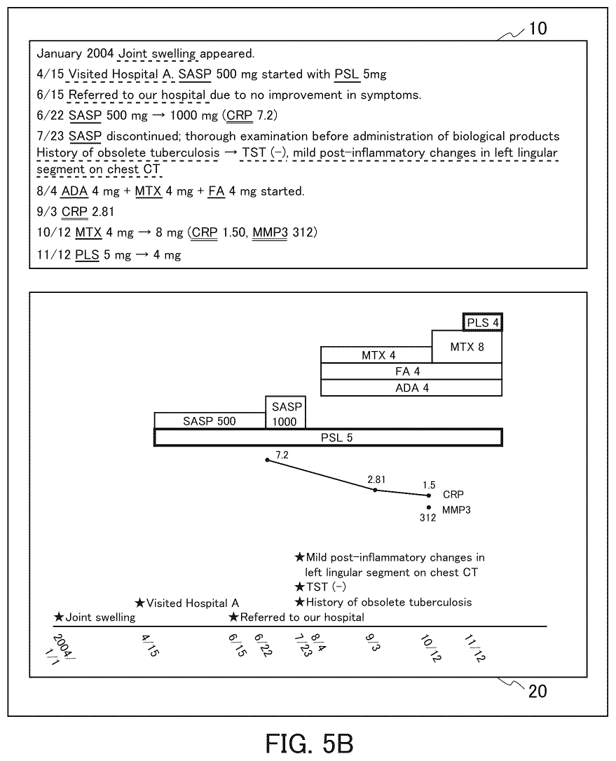

FIG. 5A shows an example of the progress diagram in a case where there is an erroneous input in the event information. In this example, it is assumed that the future date is not included. This is an example of an erroneous input that the drug “PSL” was input erroneously as “PLS” when inputting findings on 11/12. Note that, in this example, it is assumed that at least words each indicated with a single underline, a double underline, or a dashed line are registered in the dictionary information for recognition, and the word “PLS” is not registered in the dictionary information for recognition. In this case, since the progress diagram generation unit 103 does not recognize the “PLS” which is not registered in the dictionary information for recognition, “PLS” is not shown in the progress diagram displayed in the progress diagram display field 20. In addition, the progress diagram generation unit 103 does not change the shape of the stacked chart (the shape of the portion surrounded by the thick frame) showing the “PSL” in the progress diagram because the “PSL” and the “PLS” are different notations. As described above, when the progress diagram is generated using the dictionary information for recognition, the visibility is improved and the contents of the observation information are easily grasped, which allows an erroneous input to be easily found. In addition, it is possible to assist an improvement in the accuracy of the contents of the observation information.

FIG. 5B shows another example of the progress diagram in a case where there is an erroneous input in the event information. In this example, it is assumed that the future date is not included. This is an example of an erroneous input that the drug “PSL” was input erroneously as “PLS” when inputting findings on 11/12. Note that, in this example, it is assumed that at least words each indicated with a single underline, a double underline, or a dashed line are registered in the dictionary information for recognition. That is, in this example, unlike the example shown in FIG. 5A, the word “PLS” is also registered in the dictionary information for recognition. In this case, the progress diagram generation unit 103 also recognizes “PLS” which is an erroneous input. However, since “PSL” and “PLS” are different notations, a progress diagram is generated in which the position on the second axis where “PLS” is shown by a stacked chart is different from the position on the second axis where “PSL” is shown (in FIG. 5B, the positions where “PSL” and “PLS” are shown by stacked charts are surrounded by the thick frame). As described above, even if the same word as the word erroneously input is registered in the dictionary information for recognition, since the display positions on the progress diagram are different, the erroneous input can be easily found.

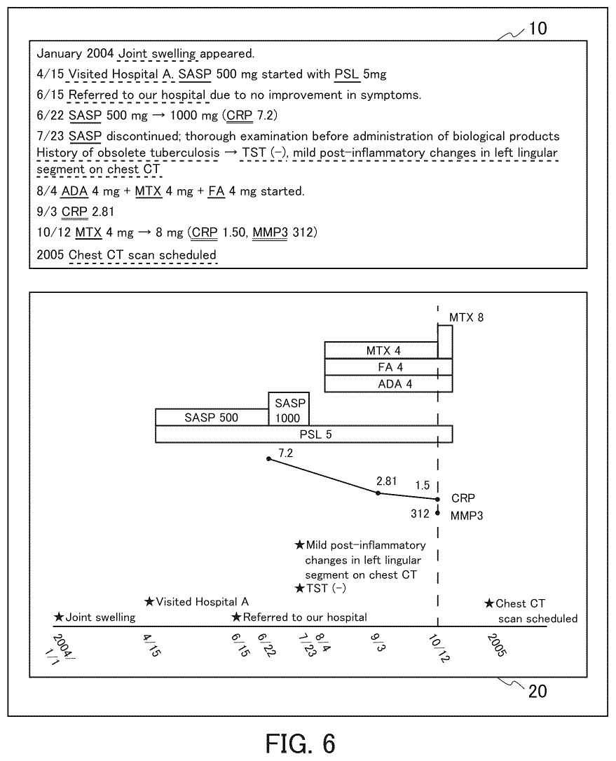

FIG. 6 shows an example of the progress diagram in a case where the future date is included. In this example, it is assumed that the execution date and time is Oct. 12, 2004. In this example, event information (qualitative information) corresponding to 2005, which is the future date, is input to the observation information. In this case, an object (indicated by a dashed line in FIG. 6) indicating the execution date and time is drawn in the progress diagram generated by the progress diagram generation unit 103. The object indicating the execution date and time (hereinafter, also referred to as the execution day line) may be a line as shown in FIG. 6, but this is an example and is not limited thereto. That is, the progress diagram includes a past area indicating the event information before the execution date and time, and a future area indicating the event information in the future later than the execution date and time, with the execution day line as a boundary. As shown in FIG. 6, the other end of the stacked object showing the quantitative information at the execution date and time (including the quantitative information continued or estimated to be continued until the execution date and time) is set to a position of at least a predetermined length from the one end as in the condition (1). In this way, even if a future date and time is input, the event information (the qualitative information) is shown at a position corresponding to the future date in the future area. That is, when a schedule such as a test is input as a preliminary record to the observation information, the schedule can be shown in the progress diagram by the apparatus 100. Further, by drawing the object indicating the execution date and time, the boundary between the event information up to the execution date and time and the event information of the future can be easily grasped.

The progress diagram generated by the progress diagram generation unit 103 may include, for example, auxiliary lines extending in the second axial direction. In the progress diagram, the auxiliary lines are drawn, for example, at regular intervals (1 month, 3 months, etc.) in the first axis.

### Third Example Embodiment

An example of the graphical user interface (GUI) in a case where an example aspect of the invention is applied to an electronic health record of a medical site is described below.

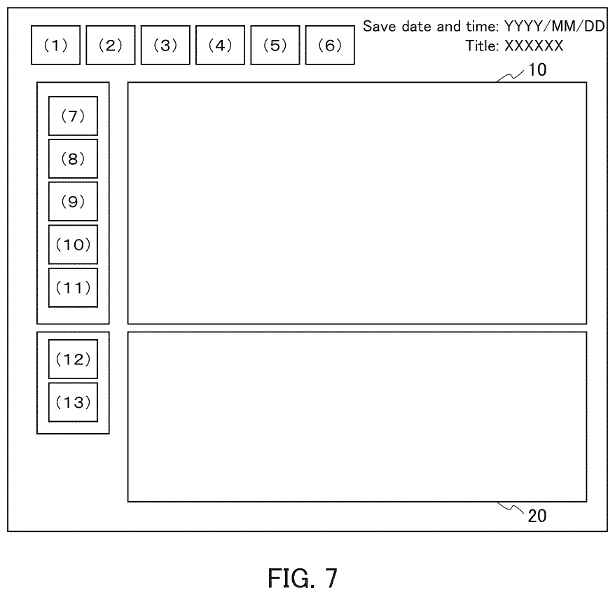

FIG. 7 is a schematic diagram showing an example of an operation screen (graphical user interface) for implementing the first and second example embodiments. As described above, in some areas of GUI, for example, provided are an input field 10 where text data that can be the observation information can be input or edited and a progress diagram display field 20 where the progress diagram generated from the text data input to the input field 10 is displayed.

As shown in FIG. 7, various types of icons (1) to (13) are arranged in a partial area of the operation screen, for example. Note that this is merely an example, and the types of icons are not limited thereto. The functions of the icons of GUI are described in the following (1) to (13). The functions described in the following (1) to (13) correspond to the functions of the icons (1) to (13) shown in FIG. 7, respectively. With the selection by the input device as a trigger, the icon provides an instruction to the central processing unit 1 to implement the following functions.

As shown in FIG. 7, for example, a toolbar is provided in a partial area of the operation screen. In the toolbar, arranged are icons for instructing a computer to implement respective functions of (1) to (6) described below.

### (1) Create

This is a function of creating new text data that becomes the observation information from a state in which no text data is input to the input field 10. This function is executed by the observation information acquisition unit 101, for example. That is, for example, the observation information acquisition unit 101 may execute not only the acquisition of the observation information but also various processes related to creation of the observation information.

### (2) Open

This is a function of opening text data that has already been saved and inputting it to the input field 10. This function is executed by the observation information acquisition unit 101, for example. The destination to save is not particularly limited, and may be the memory 2 and the storage 4, an external device, or a browser.

### (3) Dictionary

This is a function of allowing the dictionary information for recognition and the dictionary information for non-recognition to be edited. This function is executed by the registration unit 105, for example.

### (4) Save

This is a function of saving the text data input to the input field 10 as new text data. This function is executed by the observation information acquisition unit 101, for example. This function is executed by the observation information acquisition unit 101, for example. The destination to save is, for example, the same as those described above.

### (5) Overwrite Save

This is the function of overwriting and saving the correction to the text data opened and input by the function of (2). This function is executed by the observation information acquisition unit 101, for example. In the case of such overwriting, the text data is saved in the same destination to save as the destination where the text data has already been saved.

### (6) Copy

This is the function of copying the text data input to the input field 10 to the clipboard in a text format. This function is executed by the observation information acquisition unit 101, for example.

As shown in FIG. 7, for example, a display area of various dictionary tools is provided in a partial area of the operation screen. In the display area of the dictionary tools, arranged are icons for instructing a computer to implement respective functions of (7) to (11) described below. These functions are executed by the registration unit 105, for example.

### (7) Drug Name

This is a function of registering the selected word in the text data input to the input field 10 as a drug name in the dictionary information for recognition. A word registered in the dictionary information for recognition as a drug name is registered in the dictionary information for recognition in association with classification information indicating that the word is a word (quantitative information) to be shown by the stacked chart. When an example aspect of the invention is applied to other fields, for example, a word (quantitative information) to be shown by the stacked chart may be registered by this function.

### (8) Test Name

This is a function of registering the selected word in the text data input to the input field 10 as a test name in the dictionary information for recognition. A word registered in the dictionary information for recognition as a test name is registered in the dictionary information for recognition in association with classification information indicating that the word is a word (quantitative information) to be shown by the line chart. When an example aspect of the invention is applied to other fields, for example, a word (quantitative information) to be shown by the line chart may be registered by this function.

### (9) Comment

This is a function of registering the selected word in the text data input to the input field 10 as a comment in the dictionary information for recognition. A word registered in the dictionary information for recognition as a comment is registered in the dictionary information for recognition in association with classification information indicating that the word is a word (qualitative information) to be shown by a comment. When an example aspect of the invention is applied to other fields, for example, a word (qualitative information) to be shown by a comment may be registered by this function.

### (10) Delete

This is a function of deleting, from the dictionary information for recognition or the dictionary information for non-recognition, a selected word in the text data that has already been registered in the dictionary information for recognition or the dictionary information for non-recognition and input to the input field 10.

### (11) Mute

This is a function of registering the selected word in the text data input to the input field 10 in the dictionary information for non-recognition.

As shown in FIG. 7, for example, a display area of various progress diagram tools is provided in a partial area of the operation screen. In the display area of the progress diagram tools, provided are icons for instructing a computer to implement respective functions of (12) to (13) described below.

### (12) Download

This is a function of downloading the progress diagram displayed in the progress diagram display field 20 in a specific format. This function is executed by the output unit 104, for example. The specific format is the editable format described above, and specific examples of such a format include a SVG format and a BMP format. The destination to download is not particularly limited, and may be, for example, the memory 2 and the storage 4, or may be an external device.

### (13) Copy

This is a function of copying the progress diagram displayed in the progress diagram display field 20 to the clipboard in a specific format. This function is executed by the output unit 104, for example. The specific format is not particularly limited, and is, for example, the same as those described in (12) above.

For example, a checkbox for each test name shown in the progress diagram may be displayed in a partial area of the progress diagram display field 20. When the user operates the checkbox (place a checkmark in or remove a checkmark from the checkbox), the display and non-display of the test name in the progress diagram can be individually switched. While the test name has been described as an example in the present example embodiment, an example aspect of the invention is not limited thereto, and the same function can be achieved by the operation of the checkbox as to the words to be shown by the line chart (quantitative information). This function is executed by, for example, the progress diagram generation unit 103.

The operation screen as described above allows an intuitive operation by the user. In addition, the observation information and the progress diagram can be displayed on the same screen as icons for implementing various functions, which further improves the operability.

### Fourth Example Embodiment

The program of the present example embodiment is a program for causing a computer to execute processes of the method according to an example aspect of the invention as a procedure. As used herein, the term, “procedure” may be read as the “processing”. The program of the present example embodiment may be, for example, a program added in to existing software. The program of the present example embodiment may be recorded in a computer readable recording medium, for example. The recording medium is, for example, a non-transitory computer readable storage medium. The recording medium is not particularly limited, and examples thereof include read only memories (ROMs), hard disks (HDs), and optical disks.

While the invention has been particularly shown and described with reference to example embodiments thereof, the invention is not limited to these example embodiments. It will be understood by those of ordinary skill in the art that various changes in form and details may be made therein without departing from the spirit and scope of the present invention as defined by the claims.

This application is based upon and claims the benefit of priority from Japanese patent application No. 2021-214645, filed on Dec. 28, 2021, the disclosure of which is incorporated herein in its entirety by reference.

### SUPPLEMENTARY NOTE

The whole or part of the example embodiments disclosed above can be described as, but not limited to, the following supplementary notes.

### Supplementary Note 1

A progress diagram generation apparatus, including:
an observation information acquisition unit;
a time series data generation unit; and
a progress diagram generation unit, wherein
the observation information acquisition unit is configured to acquire observation information related to an observation target,
the time series data generation unit is configured to extract date and time information indicating at least one of a date or a time and event information associated with the date and time information from the observation information and structure them to generate time series data, and
the progress diagram generation unit is configured to generate a progress diagram showing the event information along a time elapsed based on the time series data.

### Supplementary Note 2

The progress diagram generation apparatus according to Supplementary Note 1, wherein
the progress diagram generation unit is configured to generate a progress diagram including a first axis indicating a time elapsed and a second axis indicating the event information based on the time series data.

### Supplementary Note 3

The progress diagram generation apparatus according to Supplementary Note 1 or 2, wherein
the observation information acquisition unit is configured to acquire image information and acquire the observation information from the image information using a character recognition technique.

### Supplementary Note 4

The progress diagram generation apparatus according to any one of Supplementary Notes 1 to 3, wherein
the progress diagram generation unit is configured to generate the progress diagram showing the event information by at least one of a stacked chart, a line chart, or a comment.

### Supplementary Note 5

The progress diagram generation apparatus according to Supplementary Note 4, wherein
the observation information includes at least one of quantitative information or qualitative information as the event information, and
the progress diagram generation unit is configured to generate the progress diagram showing the quantitative information by at least one of the stacked chart or the line chart and showing the qualitative information by the comment, based on the time series data.

### Supplementary Note 6

The progress diagram generation apparatus according to Supplementary Note 5, wherein
when the observation information includes date and time information indicating at least one of a date or a time in future later than an execution date and time of processing related to generation of the progress diagram,
an object indicating the execution date and time is drawn in the progress diagram generated by the progress diagram generation unit.

### Supplementary Note 7

The progress diagram generation apparatus according to Supplementary Note 5 or 6, further including:
a registration unit, wherein
the registration unit is configured to register a freely selected word to be recognized by the time series data generation unit and classification information in association with each other in dictionary information for recognition,
the classification information is information indicating that the freely selected word is a word classified into any one of the quantitative information to be shown by a stacked chart, the quantitative information to be shown by a line chart, or the qualitative information to be shown by a comment in a progress diagram, and
the progress diagram generation unit is configured to recognize, using the dictionary information, event information having the same word as a word registered in the dictionary information to generate the progress diagram showing the event information.

### Supplementary Note 8

The progress diagram generation apparatus according to any one of Supplementary Notes 1 to 7, further including:
an output unit, wherein
the output unit is configured to output the progress diagram in an editable format.

### Supplementary Note 9

The progress diagram generation apparatus according to any one of Supplementary Notes 1 to 8, wherein
at least one of the observation information acquisition unit, the time series data generation unit, or the progress diagram generation unit functions as an add-in or an application.

### Supplementary Note 10

A progress diagram generating method, including:
observation information acquiring;
time series data generating; and
progress diagram generating, wherein
the observation information acquiring acquires observation information related to an observation target,
the time series data generating extracts date and time information indicating at least one of a date or a time and event information associated with the date and time information from the observation information and structures them to generate time series data, and
the progress diagram generating generates a progress diagram showing the event information along a time elapsed based on the time series data.

### Supplementary Note 11

The progress diagram generating method according to Supplementary Note 10, wherein
the progress diagram generating generates a progress diagram including a first axis indicating a time elapsed and a second axis indicating the event information based on the time series data.

### Supplementary Note 12

The progress diagram generating method according to Supplementary Note 10 or 11, wherein
the observation information acquiring acquires image information and acquires the observation information from the image information using a character recognition technique.

### Supplementary Note 13

The progress diagram generating method according to any one of Supplementary Notes 10 to 12, wherein
the progress diagram generating generates the progress diagram showing the event information by at least one of a stacked chart, a line chart, or a comment.

### Supplementary Note 14

The progress diagram generating method according to Supplementary Note 13, wherein
the observation information includes at least one of quantitative information or qualitative information as the event information, and
the progress diagram generating generates the progress diagram showing the quantitative information by at least one of the stacked chart or the line chart and showing the qualitative information by the comment, based on the time series data.

### Supplementary Note 15

The progress diagram generating method according to Supplementary Note 14, wherein
when the observation information includes date and time information indicating at least one of a date or a time in future later than an execution date and time of processing related to generation of the progress diagram,
an object indicating the execution date and time is drawn in the progress diagram generated by the progress diagram generating.

### Supplementary Note 16

The progress diagram generating method according to Supplementary Note 14 or 15, further including:
registering, wherein
the registering registers a freely selected word to be recognized by the time series data generating and classification information in association with each other in dictionary information for recognition,
the classification information is information indicating that the freely selected word is a word classified into any one of the quantitative information to be shown by a stacked chart, the quantitative information to be shown by a line chart, or the qualitative information to be shown by a comment in a progress diagram, and
the progress diagram generating recognizes, using the dictionary information, event information having the same word as a word registered in the dictionary information to generate the progress diagram showing the event information.

### Supplementary Note 17

The progress diagram generating method according to any one of Supplementary Notes 10 to 16, further including:
outputting, wherein
the outputting outputs the progress diagram in an editable format.

### Supplementary Note 18

The progress diagram generating method according to any one of Supplementary Notes 10 to 17, wherein
at least one of the observation information acquiring, the time series data generating, or the progress diagram generating functions as an add-in or an application.

### Supplementary Note 19

A program for causing a computer to execute:
an observation information acquisition procedure;
a time series data generation procedure; and
a progress diagram generation procedure, wherein
the observation information acquisition procedure acquires observation information related to an observation target,
the time series data generation procedure extracts date and time information indicating at least one of a date or a time and event information associated with the date and time information from the observation information and structures them to generate time series data, and
the progress diagram generation procedure generates a progress diagram showing the event information along a time elapsed based on the time series data.

### Supplementary Note 20

The program according to Supplementary Note 19, wherein
the progress diagram generation procedure generates a progress diagram including a first axis indicating a time elapsed and a second axis indicating the event information based on the time series data.

### Supplementary Note 21

The program according to Supplementary Note 19 or 20, wherein
the observation information acquisition procedure acquires image information and acquires the observation information from the image information using a character recognition technique.

### Supplementary Note 22

The program according to any one of Supplementary Notes 19 to 21, wherein
the progress diagram generation procedure generates the progress diagram showing the event information by at least one of a stacked chart, a line chart, or a comment.

### Supplementary Note 23

The program according to Supplementary Note 22, wherein
the observation information includes at least one of quantitative information or qualitative information as the event information, and
the progress diagram generation procedure generates the progress diagram showing the quantitative information by at least one of the stacked chart or the line chart and showing the qualitative information by the comment, based on the time series data.

### Supplementary Note 24

The program according to Supplementary Note 23, wherein
when the observation information includes date and time information indicating at least one of a date or a time in future later than an execution date and time of processing related to generation of the progress diagram,
an object indicating the execution date and time is drawn in the progress diagram generated by the progress diagram generation procedure.

### Supplementary Note 25

The program according to Supplementary Note 23 or 24, further including:
a registration procedure, wherein
the registration procedure registers a freely selected word to be recognized by the time series
data generation procedure and classification information in association with each other in dictionary information for recognition,
the classification information is information indicating that the freely selected word is a word classified into any one of the quantitative information to be shown by a stacked chart, the quantitative information to be shown by a line chart, or the qualitative information to be shown by a comment in a progress diagram, and
the progress diagram generation procedure recognizes, using the dictionary information, event information having the same word as a word registered in the dictionary information to generate the progress diagram showing the event information.

### Supplementary Note 26

The program according to any one of Supplementary Notes 19 to 25, further including:
an output procedure, wherein
the output procedure outputs the progress diagram in an editable format.

### Supplementary Note 27

The program according to any one of Supplementary Notes 19 to 26, which is a program included in an add-in or an application.

### Supplementary Note 28

A non-transitory computer readable recording medium recorded with the program according to any one of Supplementary Notes 19 to 27.

### INDUSTRIAL APPLICABILITY

The present invention allows time series to be easily grasped and input contents to be easily checked. Thus, the present invention is particularly useful, for example, in the case of grasping information on a medical record.

### REFERENCE SIGNS LIST

1: central processing unit
2: memory
3: bus
4: storage
5: input device
6: output device
7: communication device
10: input field
20: progress diagram display field
100: progress diagram generation apparatus
101: observation information acquisition unit
102: time series data generation unit
103: progress diagram generation unit
104: output unit
105: registration unit

1. An apparatus for progress diagram generation, comprising at least one processor configured to:
acquire observation information related to an observation target,
extract date and time information indicating at least one of a date or a time and event information associated with the date and time information from the observation information and structure them to generate time series data, and
generate a progress diagram showing the event information along a time elapsed based on the time series data.

2. The apparatus according to claim 1, wherein the processor is configured to:
generate a progress diagram including a first axis indicating a time elapsed and a second axis indicating the event information based on the time series data.

3. The apparatus according to claim 1, wherein the processor is configured to:
acquire image information and acquire the observation information from the image information using a character recognition technique.

4. The apparatus according to claim 1, wherein the processor is configured to:
generate the progress diagram showing the event information by at least one of a stacked chart, a line chart, or a comment.

5. The apparatus according to claim 4, wherein the processor is configured to:
the observation information includes at least one of quantitative information or qualitative information as the event information, and
generate the progress diagram showing the quantitative information by at least one of the stacked chart or the line chart and showing the qualitative information by the comment, based on the time series data.

6. The apparatus according to claim 5, wherein the processor is configured to:
when the observation information includes date and time information indicating at least one of a date or a time in future later than an execution date and time of processing related to generation of the progress diagram,
an object indicating the execution date and time is drawn in the progress diagram.

7. The apparatus according to claim 5, wherein the processor is configured to:
register a freely selected word to be recognized by the time series data generation unit and classification information in association with each other in dictionary information for recognition,
the classification information is information indicating that the freely selected word is a word classified into any one of the quantitative information to be shown by a stacked chart, the quantitative information to be shown by a line chart, or the qualitative information to be shown by a comment in a progress diagram, and
recognize, using the dictionary information, event information having the same word as a word registered in the dictionary information to generate the progress diagram showing the event information.

8. The apparatus according to claim 1, wherein the processor is configured to:
output the progress diagram in an editable format.

9. The apparatus according to claim 1, wherein the processor is configured to:
a functions of an add-in or a functions of an application performs at least one of acquire the observation information, generate the time series data, or generate the progress diagram.

10. A computer-implemented method for a progress diagram generating, wherein comprising:
acquiring observation information related to an observation target,
generating extracts date and time information indicating at least one of a date or a time and event information associated with the date and time information from the observation information and structures them to generate time series data, and
generating a progress diagram showing the event information along a time elapsed based on the time series data.

11. The method according to claim 10, wherein
generating a progress diagram including a first axis indicating a time elapsed and a second axis indicating the event information based on the time series data.

12. The method according to claim 10, wherein
acquiring image information and acquires the observation information from the image information using a character recognition technique.

13. The method according to claim 10, wherein
generating the progress diagram showing the event information by at least one of a stacked chart, a line chart, or a comment.

14. The method according to claim 13, wherein
the observation information includes at least one of quantitative information or qualitative information as the event information, and
generating the progress diagram showing the quantitative information by at least one of the stacked chart or the line chart and showing the qualitative information by the comment, based on the time series data.

15. The method according to claim 13, wherein
when the observation information includes date and time information indicating at least one of a date or a time in future later than an execution date and time of processing related to generation of the progress diagram,
an object indicating the execution date and time is drawn in the progress diagram.

16. The method according to claim 14, further comprising:
registering a freely selected word to be recognized and classification information in association with each other in dictionary information for recognition,
the classification information is information indicating that the freely selected word is a word classified into any one of the quantitative information to be shown by a stacked chart, the quantitative information to be shown by a line chart, or the qualitative information to be shown by a comment in a progress diagram, and
generating recognizes, using the dictionary information, event information having the same word as a word registered in the dictionary information to generate the progress diagram showing the event information.

17. The method according to claim 10, further comprising:
outputting the progress diagram in an editable format.

18. The method according to claim 10, wherein
a functions of an add-in or a function of an application performs at least one of acquiring the observation information, generating the time series data, or generating the progress diagram.

19-27. (canceled)

28. A non-transitory computer readable recording medium recorded with the program, wherein
the program causes a computer to execute a method for progress diagram generation, wherein,
the method comprising:
acquiring observation information related to an observation target,
generating extracts date and time information indicating at least one of a date or a time and event information associated with the date and time information from the observation information and structures them to generate time series data, and
generating a progress diagram showing the event information along a time elapsed based on the time series data.
